(19) United States
(12) Patent Application Publication
(10) Pub. No.: US 2015/0342621 A1
(43) Pub. Date: Dec. 3, 2015

(54) ILLUMINATED ENDOSCOPIC PEDICLE PROBE WITH DYNAMIC REAL TIME MONITORING FOR PROXIMITY TO NERVES

(71) Applicant: Avery M. Jackson, III, Grand Blanc, MI (US)

(72) Inventor: Avery M. Jackson, III, Grand Blanc, MI (US)

(21) Appl. No.: 14/723,067

(22) Filed: May 27, 2015

Related U.S. Application Data
(63) Continuation-in-part of application No. 14/289,795, filed on May 29, 2014.

(51) Int. Cl. A61B 17/16; A61B 1/06; A61B 1/015; A61B 17/17; A61B 5/11; A61N 1/36; A61B 17/86; A61B 1/00; A61B 5/0488

(52) U.S. Cl. CPC A61B 17/1671; A61B 1/00087; A61B 1/00045; A61B 1/06; A61B 1/015; A61B 5/0488; A61B 5/1107; A61N 1/36; A61B 17/86; A61B 17/1732; A61B 17/1757; A61B 2017/0262

(57) ABSTRACT

An endoscopic pedicle probe for use during spinal surgery to form a hole in a pedicle for reception of a pedicle screw has an enlarged proximal end for cooperation with the hand of the surgeon and an elongate shaft terminating in a distal tip that may be pushed through the pedicle to form the hole. An integrated endoscope and light extend through the shaft to enable the surgeon to visually observe the target area, and a conduit extends through the shaft to convey a fluid to irrigate the target area. In a preferred form the probe is connected with an electromyographic or mechanomyographic monitoring system to alert the surgeon when a breach is about to occur. In a further embodiment, two endoscopes are associated with the probe. The complete probe may be disposable, or just the tip may be detachable for disposal or replacement.

This application is a continuation-in-part of U.S. patent application Ser. No. 14/289,795, filed May 29, 2014, which claims the benefit of U.S. provisional patent application Ser. No. 61/955,895, filed Mar. 20, 2014, and is a continuation-in-part of U.S. patent application Ser. No. 13/728,987, filed Dec. 27, 2012, which in turn claims the benefit of U.S. provisional patent application Ser. No. 61/647,747, filed May 16, 2012.

### TECHNICAL FIELD

This invention relates generally to surgical instruments. More specifically, the invention relates to a pedicle probe for use in forming holes in a vertebral pedicle in preparation for pedicle screw insertion. According to one feature of the invention the probe incorporates at least one endoscope to enable the surgeon to see the area being treated. A light is integrated with the probe to illuminate the area being treated, and in a preferred embodiment irrigation means is associated with the probe to flush debris away from the area being treated to prevent the view from being obstructed. In accordance with a further preferred embodiment the probe is provided with mechanomyography (MMG) or electromyography (EMG) capability to alert the surgeon if the pedicle is about to be breached. In another embodiment a replaceable tip is provided on the distal end of the probe, and in a still further preferred embodiment the entire probe is disposable. The probe of the invention may have any one or any combination of these features.

### BACKGROUND ART

It is sometimes necessary to perform surgery on the spine in order to repair trauma, correct a deformity, or alleviate the effects of disease. Spinal fusion or stabilization is one procedure that may be employed to treat these conditions. According to one source, at the present time there are approximately 30 million spine procedures performed globally each year, including approximately 400,000 cervical and lumbar fixations performed in the US.

Spinal fusion may be accomplished by insertion of screws into the pedicle to stabilize a spinal segment. The pedicle is a dense, stem-like structure projecting from the posterior of a vertebra, and there are two pedicles per vertebra that connect to other structures. Since the pedicle is the strongest point of attachment of the spine, significant forces can be applied to the spine without failure of the bone-to-metal connection.

To insert pedicle screws, a long, thin, metal probe is inserted through the pedicle and into the vertebral body, forming a hole for reception of the screw. Conventional pedicle probes may be straight or curved, and comprise an elongate solid metal shaft with an enlarged hand grip on the proximal end. The probe may have a shaped distal end adapted for forming a hole through the pedicle, or a separate awl or reamer may first be used to form a hole through the pedicle, and the probe then inserted into the cancellous bone of the pedicle and into the vertebral body to develop a path for the screw. A variety of probes are known in the prior art, including the so-called gear shift pedicle probe and the Fox pedicle probe. The gear shift probe has a round head on its proximal end, whereas the Fox probe has a flat disc-shaped head on its proximal end.

Most conventional modalities used to approximate or simulate screw placement are indirect, and include fluoroscopic guidance and frameless stereotactic guidance. Approximations of the pedicle and surrounding vital structures are obtained from a CT scan or MRI done prior to surgery.

Proper positioning of a conventional probe depends to an extent upon tactile feel. For instance, advancement of the probe should be smooth and consistent. A sudden plunge suggests breaking out of the pedicle laterally, and an increase in resistance indicates abutment against the pedicle or vertebral body cortex.

These conventional modalities require a steep learning curve, and improper or inaccurate manipulation of the probe and placement of the pedicle screw can result in caudal or medial penetration of the pedicle cortex and dural or neural injury.

With conventional pedicle probes there is no direct way to confirm that the hole was made within the pedicle and that the screw will be placed completely inside the pedicle. Surrounding structures can be injured if a portion of the screw is placed outside of the pedicle. There can be nerve root injury, epidural vessel injury, or spinal fluid leakage caused by a misplaced screw.

Applicant's earlier U.S. Pat. No. 6,855,105, discloses an endoscopic pedicle probe having a camera at its distal end connected with an endoscopic monitor via a fiber optic bundle extending through the probe to provide the surgeon with a view of the area being treated, thus overcoming many of the shortcomings of conventional pedicle probes.

Recognizing that illumination of the area being treated would greatly enhance the usefulness of the endoscope, in his earlier U.S. patent application Ser. No. 13/728,987, applicant added a light to illuminate the area being treated. Applicant also added irrigation means to flush debris away from the area to so that the view of the endoscope camera is not obstructed.

Notwithstanding the advantages of applicant's earlier pedicle probes, multiple bores were required through the probe shaft in order to carry the endoscope, light and irrigation means.

In applicant's prior provisional patent application Ser. No. 61/955,895, an endoscope and light are combined in a single unit that can be extended through a single bore in the probe, thus reducing the number of bores required and simplifying the construction of the probe.

Although the earlier embodiments of applicant's invention cured many of the deficiencies of prior art probes, it was difficult for the surgeon to know when a breach was about to occur due to misplacement of the probe.

U.S. Pat. No. 8,255,044 discloses a system that uses the principles of electromyography to alert the surgeon when a breach is about to occur and potentially cause damage to nerves. The system in that patent takes advantage of the insulating characteristics of the walls of the pedicle and the conductivity of adjacent nerve roots and uses electromyographic monitoring to perform dynamic pedicle integrity assessments to detect a breach or potential breach of the pedicle and alert the surgeon. The system in the '044 patent involves establishing electrical communication between a stimulation source and the interior of a pedicle hole during the hole formation, hole preparation, and/or screw introduction steps of pedicle screw fixation. By applying a stimulation signal during these steps and monitoring the neuromuscular responses resulting from this stimulation, the system automatically detects and communicates to the user whether the integrity of the pedicle has been compromised, i.e. breached or about to be breached. The probe in this patent is made of electrically conductive material and is connected with a source of electrical energy to apply an electric field to the probe. A plunger 41 is manually applied to a device 65 to establish electrical connection with the source of electrical energy. To avoid shunting between the conductive walls of the probe and adjacent tissue when the stimulation signal is applied, a flexible insulating sheath is placed around the probe body.

Recent advances in lateral access spinal fusion surgery techniques now enable surgeons to perform minimally invasive lateral access spinal fusion in a safe and effective muscle-sparing manner. Traditional posterior fusion techniques require the dissection and retraction of back muscles, bones, vessels, ligaments, and nerves; whereas traditional anterior approaches through the abdominal musculature risk injury to major vascular structures such as the aorta and iliac vessels, as well as the very delicate genitourinary structures.

In the new lateral transpsoas approach access is through the side of the patient and through the psoas muscle, using mechanomyography (MMG) to provide dynamic real-time monitoring of the location of nerves. MMG functions by measuring the mechanical response of muscle following nerve stimulation, compared to traditional electromyography (EMG) techniques that monitor the electrical response of muscle and are therefore subject to the potential for electrical interference. MMG has a faster response than EMG, indicating a higher sensitivity for detection of nerves at a lower threshold. Muscle response to electrical stimulus varies with the distance of the nerve from the source of the stimulus and MMG can tell the surgeon exactly how far away he or she is from the nerve. Working with different levels of current, the surgeon is able to establish a relationship between the current and distance, allowing the surgeon to determine precisely how far a nerve is from the stimulus probe.

MMG detects the presence of a nerve on average 1.2 seconds earlier than EMG, using approximately half the amount of stimulating current. Since electrical resistance is highly variable, depending on the conducting tissue, EMG monitoring systems may utilize currents as high as 200 mA. The MMG system typically has a maximum current output of 6 mA, nearly 35 times less than comparable EMG systems.

MMG is a more sensitive indicator for locating nerves, and the surgeon can, without looking, know within a millimeter or two where he or she is in relation to the nerve. By utilizing a system that requires less electrical current, the surgeon is able to further decrease the risk of injury to patients.

Sentio, LLC of Wixom Mich. has developed a mechanomyography (MMG) surgical access tool for locating and mapping motor nerve roots and their peripheral extensions during lateral access spinal fusion surgery. The Sentio MMG system adheres accelerometer sensors to the surface of the skin directly over muscles innervated by the nerves the surgeon wishes to identify. A stimulator probe is manipulated by the surgeon about the surgical site to stimulate for the presence of motor nerves. When a nerve is identified, the surgeon is provided with a “stop” alert. At any time the surgeon is stimulating and receiving a “go” alert, the surgeon can infer that a “go” alert when using stimulation current at:
1 mA means the Sentio probe is at least 1 mm from the nerve;
5 mA means the Sentio probe is at least 5 mm from the nerve;
15 mA means the Sentio probe is at least 15 mm from the nerve.

Sentio MMG® measures the same physiological phenomena associated with muscle contraction as EMG, but does so via mechanical means as opposed to electrical. MMG does not involve needles, therefor reducing the risk of needle sticks to the surgeon and OR staff and further reducing the chance of infection to the patient and OR personnel; does not require any skin prep; and readings require only a single sensor patch to be adhered to the skin, whereas EMG requires three electrode areas to be prepared.

In use of the Sentio system, an incision is made in the side of the patient and the surgeon inserts a dilator through the incision and to the level of the spine. A small electrical signal is sent through the dilator to stimulate nerves and guide the surgeon in placement of the dilator directly over the disc space and in front of the lumbar nerve structures. This system does not involve the use of a pedicle probe or placement of pedicle screws.

It would be advantageous to have a pedicle probe that could use mechanomyography or electromyography to stimulate and monitor neuromuscular responses during a procedure for pedicle screw placement without having to incorporate the flexible insulating shield and plunger used in the U.S. Pat. No. 8,255,044.

### SUMMARY OF THE INVENTION

In accordance with a preferred embodiment of pedicle probe according to the invention the probe is made of a non-conductive material such as carbon fiber or a strong plastic, for example, and the distal tip is made conductive by placing a conductive coating on it, for example, so that neuromuscular response can be induced at the target site by supplying electrical energy to the tip. This form of probe is disposable following use. In an alternate embodiment the probe is made of a conductive material and all but the tip is coated with a non-conductive insulating material so an electric filed is produced only at the tip. Or a conductive strip can be placed on the probe to establish a conductive path from the proximal end to the distal end. In use, particularly of an MMG system such as that by Sentio LLC, a connector such as an alligator clip can be attached to the probe so that the Sentio system can supply electrical energy to the probe. The invention provide the surgeon with a warning when a nerve is approached or a breach is about to occur so that the surgeon can adjust the position of the probe and avoid a breach and/or contact with a nerve.

In a still further embodiment the distal tip of the probe is threaded or otherwise securely removably attached to the forward or distal end of the shaft of the probe so that the tip can be replaced when worn or damaged or when a tip having different characteristics is desired. Applicant's earlier application Ser. No. 13/728,987 added a replaceable tip enabling a new or different tip to be used without having to replace the entire instrument.

As in previous embodiments, a light and endoscope are incorporated together in a single unit, thus requiring only a single bore extending longitudinally through the probe to accommodate these two features. The endoscope and light provide the surgeon with a visual indication of the position of the probe relative to the pedicle and surrounding structure during a surgical procedure, enabling the surgeon to directly confirm the location of the probe and ensuring accurate placement of the hole for receiving a pedicle screw.

Irrigation means associated with the probe flushes the area being treated with a fluid, such as, e.g. saline, to remove body fluids and debris that might otherwise obscure the view.

One suitable endoscope incorporating a light is the Medigus LEDprobe, an integrated camera and illumination device available from Medigus, Ltd. of Omer, Israel. The Medigus LEDprobe is a 1.8/2.0 mm diameter rigid endoscope which includes a 1.2 mm camera in the distal tip of the device. It is equipped with high quality 100°/140° field of view (FOV) optics and a large LED located in the handle of the device. The device has a stainless steel shaft and illumination is led through the shaft towards the distal tip of the device where the camera is located via fiber-for-illumination. The LED is powered by the video processor and, therefore, no additional peripherals are required other than a monitor. The camera used with this system has a diameter of only 1.2 mm and a length of only 5 mm. It has high quality 100 degree FOV optics and a shielded camera cable with a metal connector as well as a video processor.

In one example of the invention, a Fox probe is modified to have a hollow shaft and a small endoscope is placed in the hollow shaft, with a camera positioned at the distal end and connected via a fiber optic bundle with an endoscopic monitor to afford either a 0°, a 45° or a 90° view, depending upon the lens employed and the positioning of the camera.

The endoscopic pedicle probe of the invention puts the surgeon “in the pedicle” with the use of endoscopy and avoids breaches by using electromyography. The positioning of the probe can be directly and accurately determined during surgery, and there is no question as to whether the screw will be too medial, lateral, cranial, caudal, or deep. The surgeon will know if the wall of the pedicle is about to be breached, and the position of the probe can be adjusted to avoid a breach. The surgeon can also avoid parallax that may cause errors when using fluoroscopic guidance.

The probe of the invention will not represent an additional instrument needed for pedicle screw placement. Accordingly, there will be no additional costs or equipment needed to perform the standard spinal fusion.

The probe of the invention can be utilized in the cervical spine for lateral mass screw placement, pedicle screw placement, or trans-articular screw placement. It can be used in the thoracic, lumbar, and sacral spine for pedicle screw placement and trans-laminar screw placement, and can be used in standard open spine fusion or in minimally invasive percutaneous spine fusion.

### BRIEF DESCRIPTION OF THE DRAWINGS

The foregoing as well as other objects and advantages of the invention will become apparent from the following detailed description when considered in conjunction with the accompanying drawings, wherein like reference characters designate like parts throughout the several views, and wherein:

FIG. 1 is a somewhat schematic isometric view of a first embodiment of endoscopic pedicle probe according to the invention, shown in combination with an endoscopy monitor.

FIG. 2 is a longitudinal sectional view of the pedicle probe of FIG. 1.

FIG. 3 is a side view in elevation of a preferred embodiment of endoscopic pedicle probe according to the invention.

FIG. 4 is a side view in elevation of the probe of FIG. 3, taken at 90° to the view in FIG. 3.

FIG. 5 is an enlarged view in elevation of the endoscopic pedicle probe of FIG. 3, looking in the direction of the arrow 5.

FIG. 6 is an enlarged view of the distal end of the probe of FIGS. 3 and 4, looking in the direction of arrow 6.

FIG. 7 is a longitudinal sectional view of the probe of FIGS. 3-6, taken along line 7-7 in FIG. 5.

FIG. 8 is a longitudinal sectional view of the probe of FIGS. 3-6, taken along line 8-8 in FIG. 5.

FIG. 9 is an enlarged fragmentary view in elevation of the distal end of the probe of FIGS. 3-8.

FIG. 10 is a fragmentary end view of the probe of FIG. 9, looking in the direction of the arrow 10.

FIG. 11 is a further enlarged fragmentary view in side elevation of the area indicated by bracket 11 in FIG. 9.

FIG. 12 is an axial view of a pedicle.

FIG. 13 is a sagittal view of a pedicle.

FIGS. 14 and 15 are fragmentary perspective views depicting how a pedicle probe is used to form a hole in a pedicle.

FIGS. 16-19 are somewhat schematic views depicting the progressive steps in forming a hole in the pedicle using the pedicle probe.

FIG. 20 is a longitudinal sectional view of an embodiment of pedicle probe according to the invention, wherein a conduit for an irrigating fluid and two endoscopes with integrated lights extend through the probe.

FIG. 21 is an end view in elevation of the probe of FIG. 20, looking in the direction of the arrow 21 in FIG. 20.

FIG. 22 is a side view, shown partially in section and partially in elevation, of a further modified probe according to the invention, wherein channels are formed in the outer surface of the probe shaft and feeds for the illuminated endoscope and flushing liquid are placed in these channels, with a surrounding sleeve placed over the shaft to secure the parts in place.

FIG. 23 is a transverse sectional view taken long line 23-23 in FIG. 22.

FIG. 24 is a transverse sectional view taken long line 24-24 in FIG. 22.

FIG. 25 is an exploded isometric view, with portions broken away, of the probe and sleeve of FIGS. 22-24.

FIG. 26 is a fragmentary enlarged longitudinal sectional view of the distal end of a further embodiment of the invention wherein a removable tip is held in place by a set screw and is aligned in proper position by a keyway.

FIG. 27 is a transverse view in section taken along line 27-27 in FIG. 26.

FIG. 28 is a transverse view in section taken along line 28-28 in FIG. 26.

FIG. 29 shows a system incorporating means for applying an electrical stimulus to nerves to cause a measurable muscle contraction indicative of the proximity of the probe to a nerve during use of the probe.

FIG. 30 is an enlarged longitudinal sectional view of the probe used in the system of FIG. 29, showing an electrically conductive coating on the distal tip.

FIG. 31 is a longitudinal sectional view of an alternate embodiment of probe that can be used in the system of FIG. 29, wherein the shaft and distal tip of the probe are made of an electrically conductive material such as, e.g., steel, and all but the distal tip is coated with an insulating material to avoid unwanted shunting between the body of the probe and surrounding tissues and fluids during delivery of electrical stimulation.

FIG. 32 is a fragmentary side view in elevation of a simple ON-OFF pressure switch that can be used in the switch 95 of FIG. 31.

### BEST MODES FOR CARRYING OUT THE INVENTION

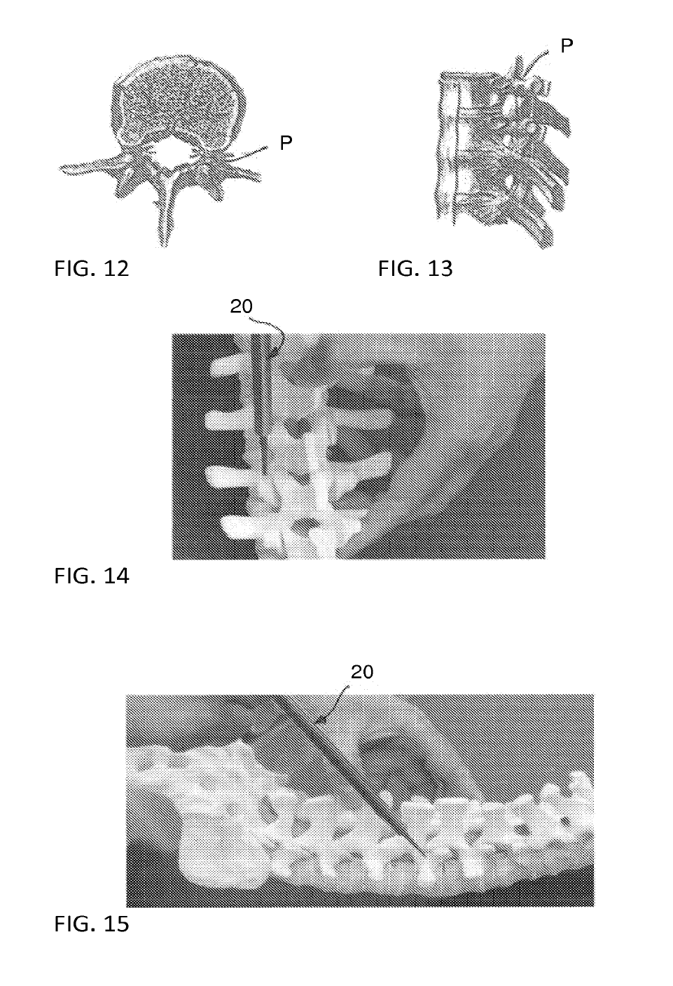

Referring more specifically to the drawings, a pedicle probe according to a first embodiment of the invention is depicted at 10 in FIG. 2. The probe has a disc-shaped head 11 on its proximal end that is about two inches in diameter, and a metal shaft 12 projecting from the center of one side thereof. A reduced diameter tip 13 on the distal end of the shaft is configured to act as a reamer, i.e., it may have a fluted configuration as found on drill bits. In use, a surgeon places the disc-shaped head 11 in the palm of his or her hand, with the shaft extending forwardly. The tip is then pushed against the pedicle while the probe is being rotated back and forth about the longitudinal axis of the shaft to form a hole in the pedicle for reception of a pedicle screw. See, for example, FIGS. 12-19.

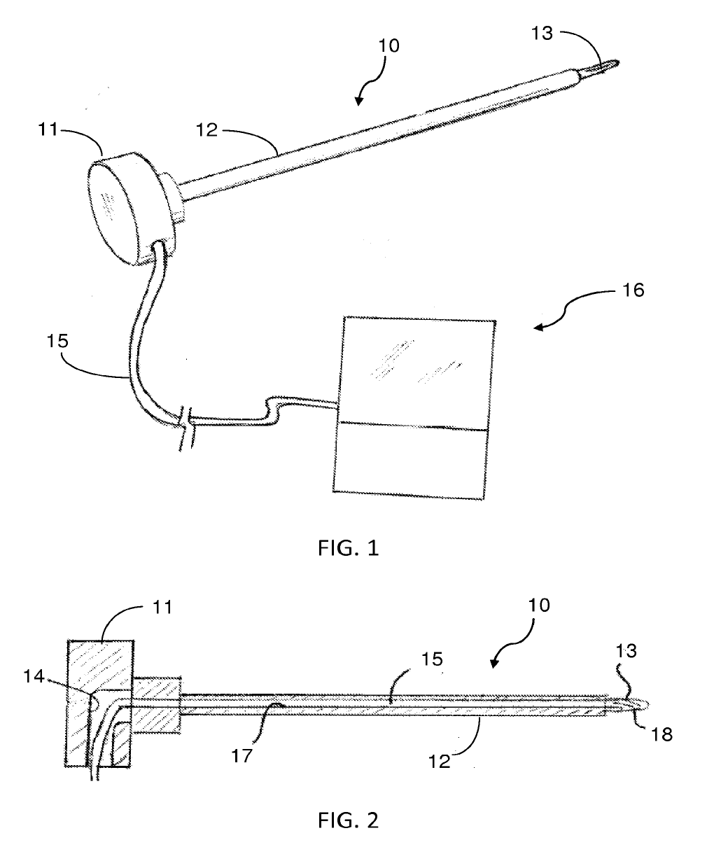

In the embodiment shown in FIGS. 1 and 2, the disc-shaped head 11 of the probe 10 has an opening 14 formed in it for receipt of an endoscope 15 with integrated illumination means, such as the Medigus LEDprobe, an integrated camera and illumination device available from Medigus, Ltd. of Omer, Israel. The endoscope is connected with a suitable conventional monitor 16. The shaft 12 has a bore 17 formed through its length for receipt of the endoscope 15, which terminates in the tip 13 at a camera 18. The tip 13 is adapted to penetrate the hard bony tissue of a vertebral pedicle to form a hole for reception of a pedicle screw.

As shown in the drawings, the tip has a substantially uniform diameter through a portion of its length, and terminates in a sharpened point. The diameter of the tip is approximately the same as, or smaller than, the diameter of a pedicle screw to be inserted in the hole formed with the probe, and will form an elongate hole having a uniform diameter for secure engagement with a screw inserted in the hole. The tip has a hardness and configuration to act as a reamer, and may have a fluted configuration as incorporated, for example, in a conventional Fox pedicle probe, to facilitate penetration of the probe through the cancellous bone.

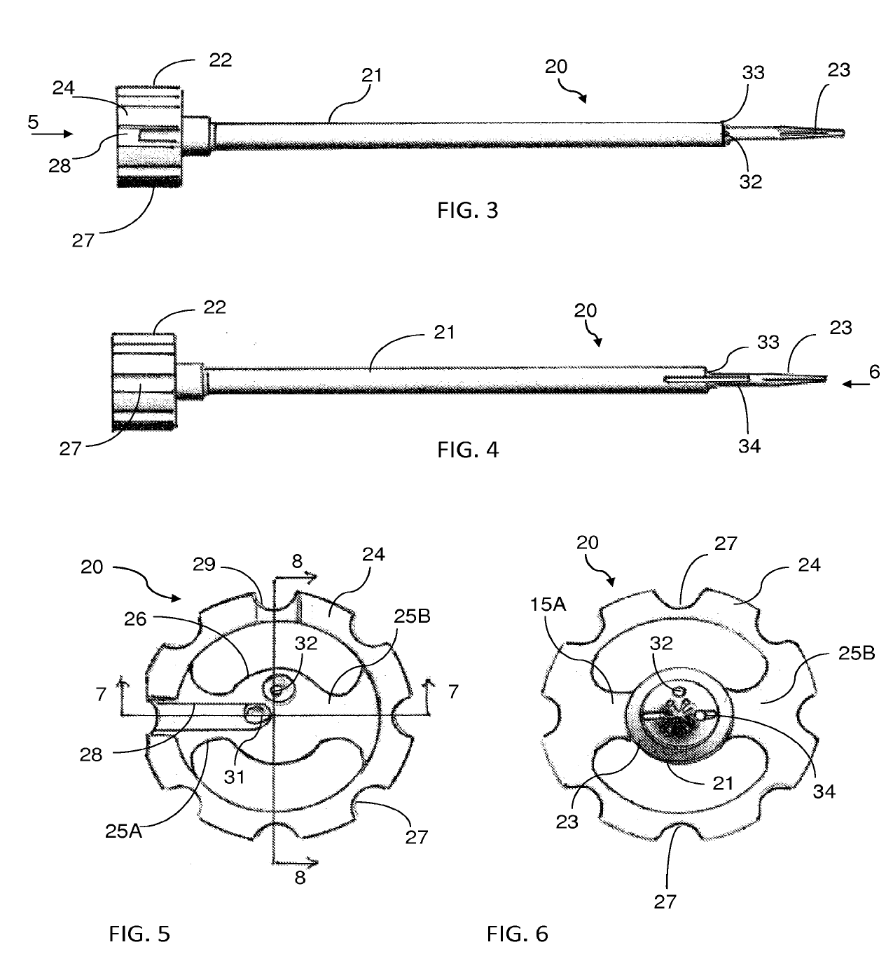

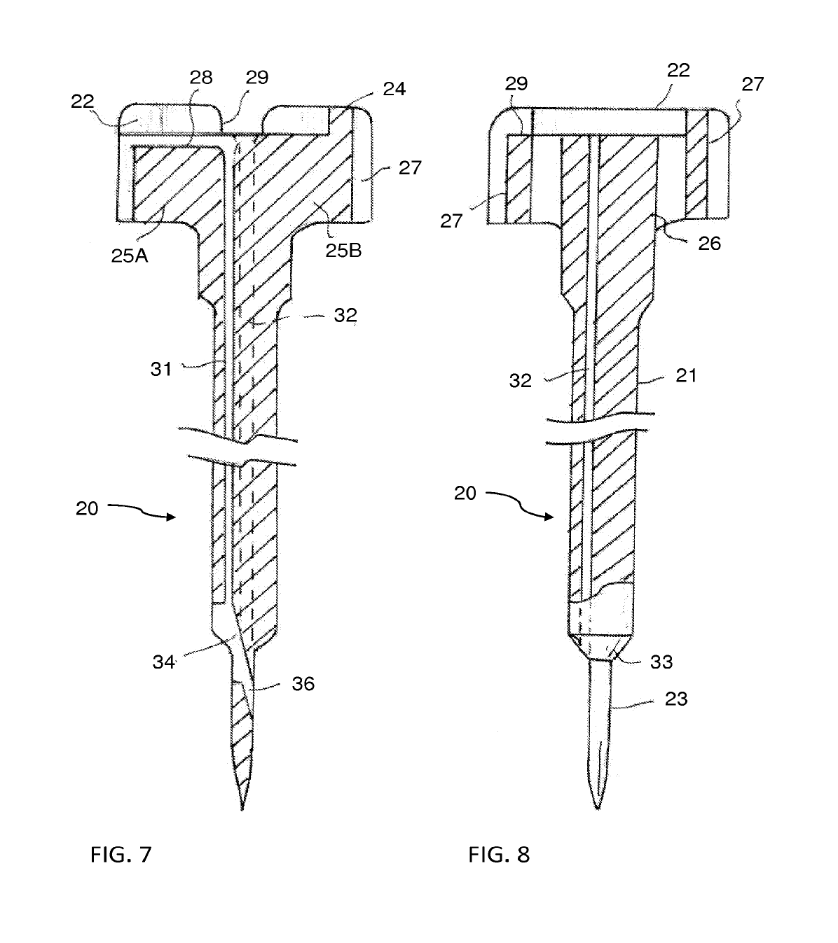

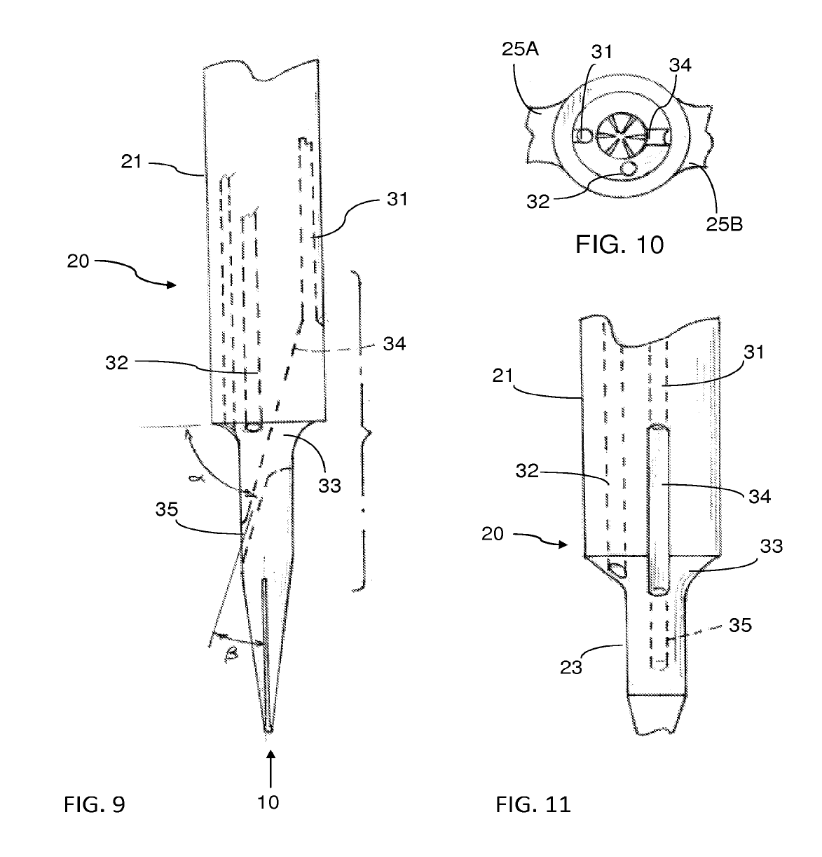

A second embodiment of endoscopic pedicle probe according to the invention is indicated generally at 20 in FIGS. 3-11. Although not shown, an endoscope with integrated illumination means as described in connection with the FIG. 1 embodiment, i.e. the Medigus LEDprobe, may also be used in this form of the invention. This form differs from that shown in FIG. 1 in that the tip 23 can be configured to position the camera 25 for providing a 90° view or a 45° forward view or a 0° view straight ahead. Thus, by selection of an appropriate probe, or by appropriate manipulation of a probe, the surgeon can obtain a direct visual indication of the exact position of the probe in the pedicle and of the pedicle itself and surrounding structure. As depicted in these figures, the camera is placed rearwardly of the distal point of the end to protect it when the probe is pressed against and pushed through hard bony tissue. An obturator, not shown, may be provided to close the opening through the side of the tip and protect the camera 25.

The probe 20 comprises an elongate shaft 21 with an enlarged generally disc-shaped head 22 on its proximal end for grasping by the surgeon as in the previous embodiments, and the reduced diameter tip 23 extending coaxially from the distal end of the shaft. The head comprises a circumferential rim 24 connected to the proximal end of the shaft 21 by at least two spokes 25A and 25B that join the rim with a hub 26 on the proximal end of the shaft. The outer surface of the rim is longitudinally fluted at 27, and two circumferentially spaced recesses 28 and 29 are formed in the upwardly facing proximal end surface 30 of the rim. As used herein, “upwardly facing” refers to the orientation when the probe is in its operative position during use. In the particular construction shown, the recesses are in alignment with respective flutes 27 and are circumferentially spaced apart 90 degrees.

Two bores 31 and 32 are formed longitudinally through the hub and shaft in positions oriented respectively on a radius extending through a respective recess 28 or 29. One of the bores 32 exits the distal end of the shaft in an axially forwardly facing shoulder 33 between the base end of the tip and the adjacent end of the shaft. The other bore 31 ends in a slot 34 formed in the side of the shaft at its distal end and extending angularly at an angle β of 16 degrees relative to the longitudinal axis of the shaft into the base end of the tip. A bore 35 extends from the slot and diagonally through the tip to exit an opposite side of the tip, providing a view extending over an angle α of 74 degrees.

The endoscope extends through the bore 31, and the other bore 32 is connected with a suitable source of an irrigating fluid, such as saline, for example, to flush debris away from the area being treated and prevent the view of the camera from being obscured.

In use, the light illuminates the pedicle in the area being treated to provide the surgeon with enhanced visibility of the area as observed through the camera 25 of the endoscope 22.

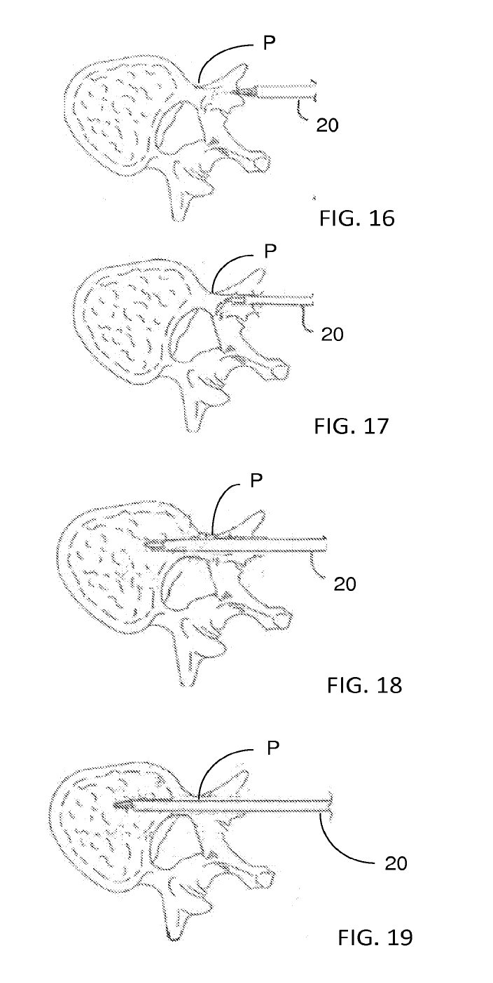

FIGS. 12 and 13 are axial and sagittal views, respectively, of a pedicle P, and FIGS. 14-19 are schematic illustrations of how a probe 20 might be used to form a hole in the pedicle. Thus, and as seen especially in FIGS. 16-19, the probe is pushed through the pedicle to form a hole for reception of a pedicle screw (not shown). Depending upon the structure of the tip 13 the probe also may be rotated back and forth to assist in forming the hole. Great care must be exercised to insure that the probe stays within the pedicle and does not break through the wall, or does not go too deep.

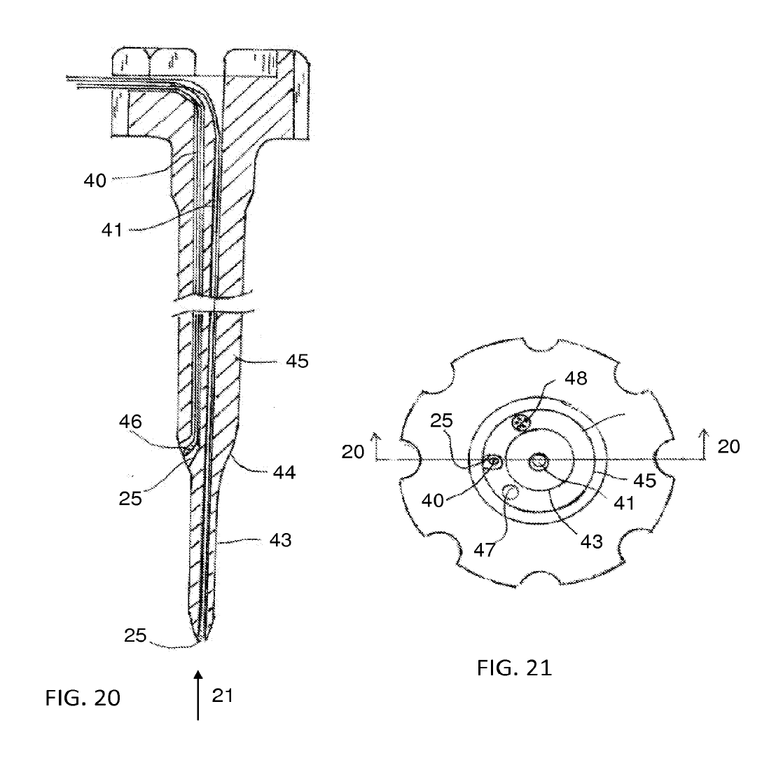

A third embodiment of probe according to the invention is shown in FIGS. 20 and 21. In this embodiment, two endoscopes 40 and 41 are provided in the probe. One of the endoscopes 41 has its camera 25 positioned at the distal end of the tip 43 in a zero degree forwardly facing orientation. The other endoscope 40 has its camera 25 positioned at the distal end 44 of the probe shaft 45 and oriented in a camera window slot 46 to provide about a 70° view looking at the side of the pedicle wall. The endoscopes 40 and 41 preferably have an illuminating means integrated with them, as in the Medigus LEDprobe discussed above, and/or a separate light 47 may be provided. A saline rinse port 48 also preferably is provided at the distal end of the probe shaft to rinse away debris during use of the probe to keep the field of vision clear.

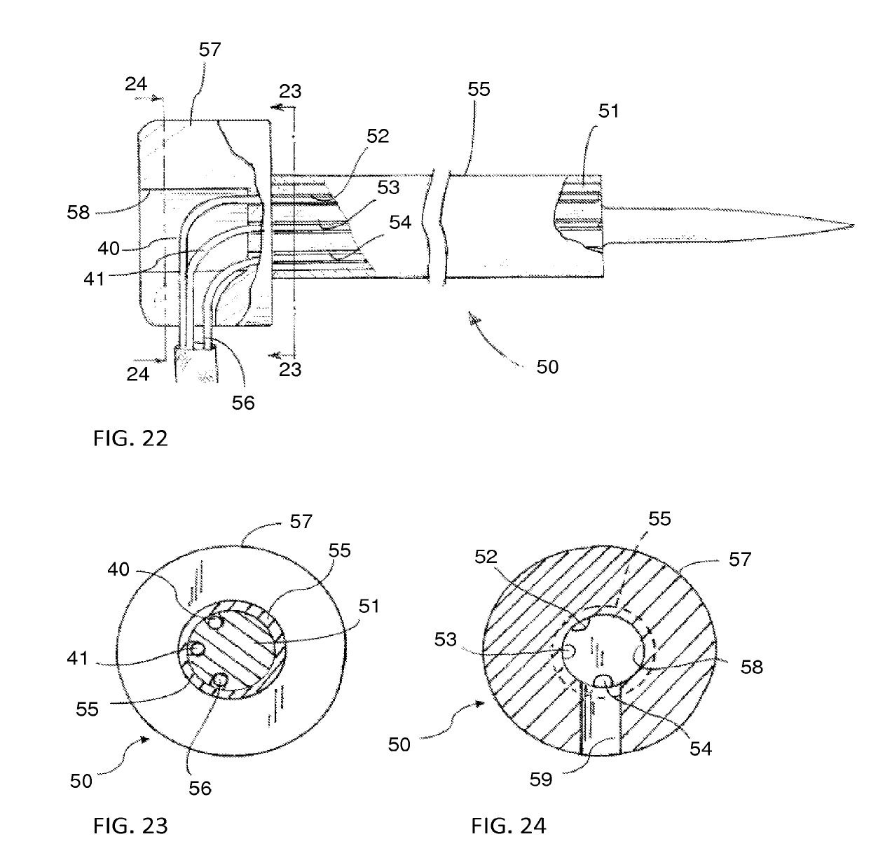

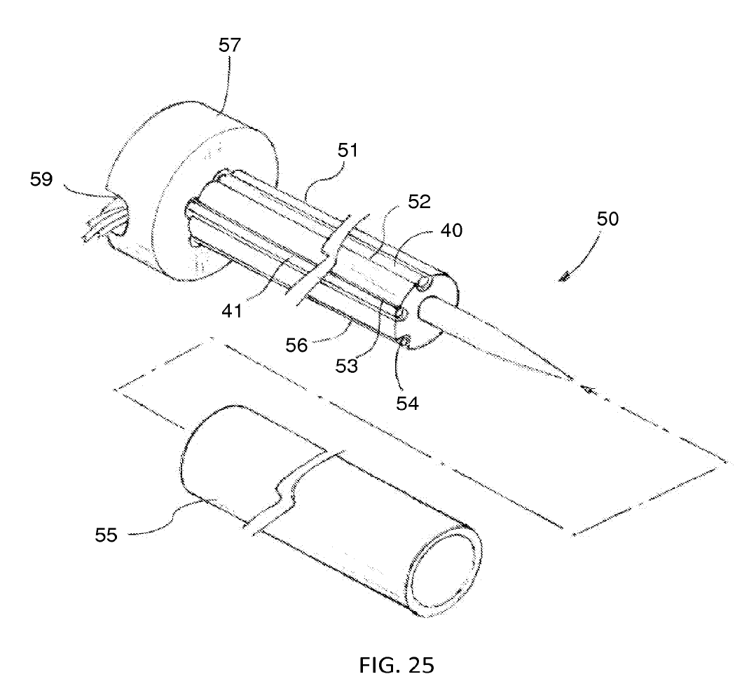

A fourth embodiment 50 of pedicle probe according to the invention is shown in FIGS. 22-25. In this form of the invention, rather than extend bores longitudinally through the body of the shaft 51 for containing the endoscope and conveying irrigation fluid, longitudinally extending channels 52, 53 and 54 are formed in the outer surface of the shaft 51, and a cylindrical sleeve 55 is positioned in snug relationship over the shaft in enclosing relationship to the channels and the endoscopes 40, 41 and conduit 56 for irrigation fluid held, respectively, therein. As in the previous embodiments, the tip may be integral with the shaft or detachable. The head 57 has a central opening 58 and the endoscopes 40, 41 and flushing conduit 56 are fed through a notch 59 in the head.

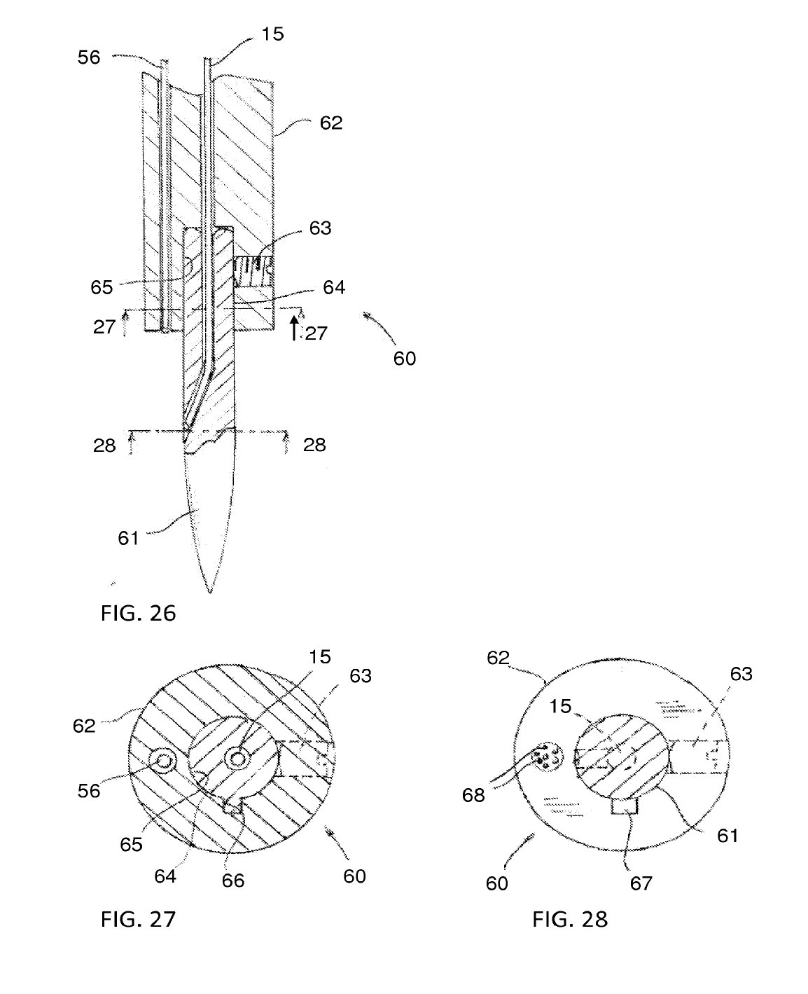

FIGS. 26-28 depict an embodiment 60 of pedicle probe in which the tip 61 is removable. In this form of the invention, the tip 61 is detachably secured to the distal end of the shaft 62 by a set screw 63 extended through the side of the shaft and into engagement with the base end 64 of the tip, which is inserted into an axial bore 65 in the distal end of the shaft. Proper rotational positioning of the tip relative to the shaft is achieved by a keyway formed by an axially extending groove or slot 66 in the interior surface of bore 65 and a complementally shaped key 67 on the exterior of the base end 64 of the tip. The tip may be readily detached from the shaft by loosening the set screw and may be used with any of the previous forms of the invention. An endoscope 15 and a conduit 56 for flushing fluid may be used in combination with the detachable tip, or the detachable tip may be used without any of these. As seen in FIG. 28, the outlet for the flushing fluid may have numerous small orifices 68 to provide a “soft” flow of flushing fluid against the area being treated.

Electromyography (EMG) or mechanomyography (MMG) may be used with the probe to alert the surgeon when a nerve is approached or a breach is about to occur. An MMG system generally is regarded as having a faster response and a higher sensitivity for detection of nerves at a lower threshold than does EMG. A suitable MMG system can be the Sentio MMG system available from Sentio LLC of Wixom, Mich.

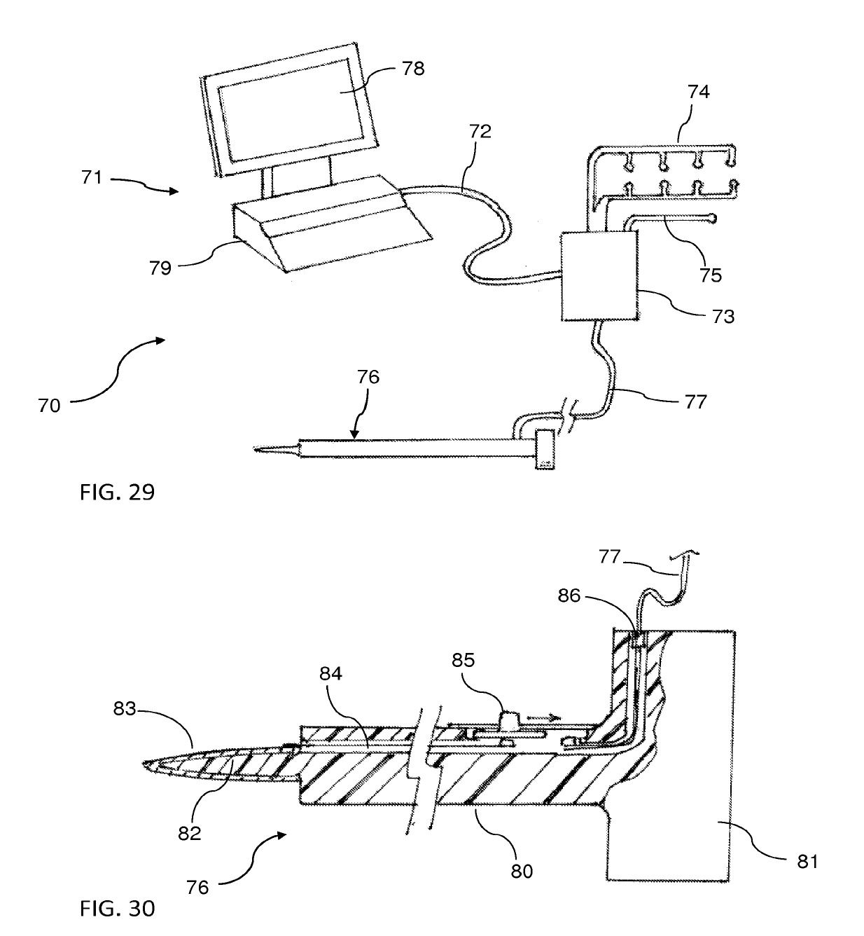

A system as it might be constituted when using either a mechanomyographic (MMG) monitoring system or an electromyographic monitoring (EMG) monitoring system is represented schematically at 70 in FIG. 29. The system would typically include a control unit 71 connected via a data cable 72 with a patient module 73. An EMG or MMG harness 74 and return electrode 75 are connected with the patient module, and a pedicle probe 76 according to the preferred form of the invention is also connected to the patient module via an electrical lead 77. The invention capitalizes on the insulating characteristics of bone, specifically, that of the medial wall of the pedicle, and the conductivity of the adjacent nerve roots. That is, if the medial wall of the pedicle is breached or in danger of being breached, i.e., the layer of bone is too thin to provide enough insulation to prevent stimulation of adjacent nerves, a stimulation signal applied to the target site will cause the various muscle groups coupled to the nerve roots to react. The employment of electromyographic or mechanomyographic monitoring in the present invention to assess whether the muscle groups in the leg are innervating in response to the application of a stimulation signal does not require visual observation of twitching of the nerves.

In the case of an EMG system, the harness 74 relies on needles to detect subtle changes in electrical signals in muscle. In contrast, a mechanomyographic system such as the Sentio MMG® system employs proprietary accelerometer technology in the harness 74. These non-invasive accelerometer-based sensors measure MMG (mechanomyography) activity, or the mechanical “twitch” associated with muscle contraction.

With either MMG or EMG the control unit 71 includes a touch screen display 78 and a base 79, which collectively contain the essential processing capabilities for controlling the system 70. The data cable 72 establishes digital and/or analog electrical connections and communications between the control unit 71 and patient module 73. The main functions of the control unit 71 include receiving user commands via the touch screen display 78, activating stimulation, processing signal data according to defined algorithms as known in U.S. Pat. No. 8,255,044, for example, displaying received parameters and processed data, and monitoring system status and reporting fault conditions. The touch screen display 78 is preferably equipped with a graphical user interface (GUI) capable of communicating information to the user and receiving instructions from the user. The display 78 and/or base 79 may contain patient module interface circuitry that commands the stimulation sources, receives digitized signals and other information from the patient module 73, processes the EMG or MMG responses to extract characteristic information for each muscle group, and displays the processed data to the operator via the display 78.

In accordance with a first preferred form of the invention as shown in FIG. 30, the probe 76 comprises an elongate shaft 80 having a disc-shaped head 81 on the proximal end and a reduced diameter tip 82 on its distal end. This form of the invention is made to be disposable following a single use and the entire probe, including the shaft and distal tip, are made of a non-conductive material such as carbon fiber or a strong plastic, for example. To enable a neuromuscular response to be stimulated at the target site when electrical energy is supplied to the probe, the tip is made electrically conductive, by coating it with an electrically conductive material 83, for example, and an electrical lead 84 is extended through the shaft and conductively connected with the coating 83. Alternatively, a conductive strip (not shown) can be extended along the outside of the probe from just below the head 81 to the coating 83. The coating may be applied using any suitable conventional method, including electrodeposition or electroless plating that enables conductive metallic materials to be coated on non-conductive materials such as plastic.

A suitable ON-OFF switch 85 preferably is provided in the electrical lead 77 or on the side of the probe shaft 80 in position to be easily accessible by the surgeon to establish or interrupt the flow of electrical energy to the tip, as desired. The switch can be a simple spring loaded slide switch that normally is biased into an open position and can be closed by engaging it with the thumb or a finger and sliding it to the appropriate position. Alternatively, the switch could be normally biased into a closed position and moved by the surgeon to an open position when desired. Further, the switch could automatically latch in either of its positions when the slide is at its limit of travel in either direction, and could be released by pressing it inwardly to disengage the latch. A disconnect 86 may be provided on the probe to enable the lead 77 to be detached from the probe when desired.

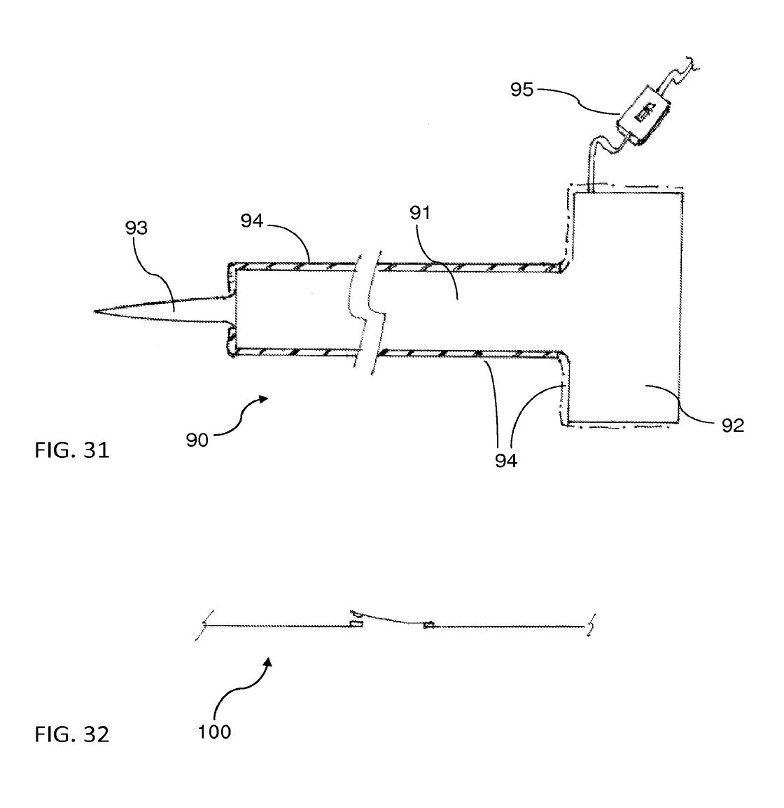

In an alternate embodiment as shown in FIG. 31, the probe 90, including the shaft 91, head 92, and distal tip 93, is made of an electrically conductive material such as steel, for example, and a coating 94 of electrical insulating material is applied to the shaft 91 and head 92 to prevent shunting when a stimulation signal is applied. The distal tip 93 is left exposed so that a stimulation signal can be applied to the target site. In this form of the invention, an electrical lead does not need to extend longitudinally through the shaft, and the lead 72 can be connected to the shaft at its proximal end. In this form of the invention the ON-OFF switch 95 preferably is positioned in the lead 72 where it can be easily accessed by the surgeon.

Another switch that could be used in any of the embodiments in lieu of the slide switch is a simple pressure switch 100 such as that shown in FIG. 32, for example, but any suitable switch means could be used. For example, a rotatable or sliding switch could be used that enables the surgeon to adjust the strength of the stimulation signal supplied to the probe.

The system 70 is capable of performing pedicle integrity assessments in a dynamic manner, that is, during the formation and/or preparation of the pilot hole and/or during pedicle screw placement. The system accomplishes this by having the control unit 71 and patient module 73 cooperate to send stimulation signals to the probe. Depending upon the effect on the bone forming the pedicle of pilot hole formation, pilot hole preparation and/or pedicle screw introduction, the stimulation signals may cause nerves adjacent to or in the general proximity of the target site to innervate, which, in turn, can be monitored via the EMG or MMG harness 74. The pedicle integrity assessment feature of the present invention is based on assessing the evoked response of the various muscle myotomes monitored by the surgical system 70 via the EMG or MMG harness 74.

In a typical example of a probe made in accordance with the invention the shaft can have a length of about 28 cm and a diameter of from about 6 mm to about 12 mm; the tip can have a length of about 40 mm and a diameter of from 4 to about 5 mm; and the endoscope 20 and conduit 56 for irrigation each can have a diameter of from about 1 mm to about 2 mm. In those embodiments wherein the camera for the endoscope is located adjacent the proximal end of the tip, it can be placed along the tip a distance spaced approximately 6 to 8 mm from where the tip is joined to the end of the shaft, and preferably is oriented at an angle of 45 to 90 degrees relative to the longitudinal axis of the tip. It should be noted that these are exemplary dimensions only and the probe and its components could have other dimensions as necessary or desirable.

The endoscopic pedicle probe of the invention provides the surgeon with an illuminated, direct visual indication of the exact location of the probe and alerts the surgeon if a breach has occurred or is about to occur. It provides for flushing body fluids and debris away from the area being treated, whereby the hole can be formed with accuracy and precision.

All of the pedicle probes disclosed herein may be reusable, or the entire probe, inclusive or not inclusive of the endoscope, may be made disposable following a single use. Materials suitable for this purpose, such as hard plastics or carbon fiber, for example, may be used in the construction of the probe.

While particular embodiments of the invention have been illustrated and described in detail herein, it should be understood that various changes and modifications may be made to the invention without departing from the spirit and intent of the invention as defined by the scope of the appended claims.

What is claimed is:

1. An endoscopic pedicle probe for use during spinal surgery to form a hole in a pedicle for reception of a pedicle screw, comprising:
an elongate shaft terminating in a distal tip that may be pushed through the pedicle to form the hole, and an enlarged proximal end for cooperation with the hand of the surgeon; and
an integrated endoscope and light extending through the shaft, said endoscope being connected with a monitor to enable a surgeon to visually observe the area being treated.

2. The endoscopic pedicle probe as claimed in claim 1, wherein:
means is provided on the probe for conveying an irrigating fluid to the site being treated to flush debris away from the area.

3. The endoscopic pedicle probe as claimed in claim 2, wherein:
the probe is connected in a system for one of electromyographic and mechanomyographic monitoring to perform dynamic pedicle integrity assessments to detect a breach or potential breach of the pedicle and alert the surgeon.

4. The endoscopic pedicle probe as claimed in claim 3, wherein:
the probe is made of non-conductive material and the distal tip is made electrically conductive so that an electrical stimulation signal supplied to the tip will innervate nerves adjacent the target site and invoke a neuromuscular response in associated muscles.

5. The endoscopic pedicle probe as claimed in claim 4, wherein:
the entire probe, including the shaft, enlarged proximal end, and distal tip are made of one of carbon fiber and plastic;
an electrical lead extends through the shaft from the proximal end to the distal end; and
the tip is coated with an electrically conductive material, said coating being connected with said lead so that a stimulation signal supplied through said lead produces the stimulation signal at said tip.

6. The endoscopic pedicle probe as claimed in claim 4, wherein:
the shaft and distal tip of said probe are made of an electrically conductive material;
an electrical lead is connected with said probe to supply a stimulation signal thereto; and
said shaft is coated with an insulating material to prevent shunting between said shaft and surrounding tissue when a stimulation signal is supplied to said probe, said tip being uncoated with insulating material so that a stimulation signal supplied to said probe produces the stimulation signal at said tip.

7. The endoscopic pedicle probe as claimed in claim 1, wherein:
two endoscopes extend through the probe, said endoscopes being positioned to provide different views of the area being treated and connected with a monitor to enable a surgeon to visually observe the area.

8. An endoscopic pedicle probe for use during spinal surgery to form a hole in a pedicle for reception of a pedicle screw, comprising:
an elongate shaft terminating in a distal tip that may be pushed through the pedicle to form the hole, and an enlarged proximal end for cooperation with the hand of the surgeon;
an endoscope and light extending through the shaft, said endoscope being connected with a monitor to enable a surgeon to visually observe the area being treated; and
an electromyographic monitoring system connected with the probe to perform dynamic pedicle integrity assessments during a procedure to detect a breach or potential breach of the pedicle and alert the surgeon.

9. The endoscopic pedicle probe as claimed in claim 8, wherein:
irrigation means is associated with the probe to convey an irrigating fluid to the site being treated to flush debris away from the site and prevent the view of the endoscope from being obscured.

10. The endoscopic pedicle probe as claimed in claim 8, wherein:
the probe is made of non-conductive material and the distal tip is made electrically conductive so that an electrical stimulation signal supplied to the tip will innervate nerves adjacent the target site and invoke a neuromuscular response in associated muscles.

11. The endoscopic pedicle probe as claimed in claim 10, wherein:
the entire probe, including the shaft, enlarged proximal end, and distal tip are made of one of carbon fiber and plastic;
an electrical lead extends through the shaft from the proximal end to the distal end; and
the tip is coated with an electrically conductive material, said coating being connected with said lead so that a stimulation signal supplied through said lead produces the stimulation signal at said tip.

12. The endoscopic pedicle probe as claimed in claim 8, wherein:
the shaft and distal tip of said probe are made of an electrically conductive material;
an electrical lead is connected with said probe to supply a stimulation signal thereto; and
said shaft is coated with an insulating material to prevent shunting between said shaft and surrounding tissue when a stimulation signal is supplied to said probe, said tip being uncoated with insulating material so that a stimulation signal supplied to said probe produces the stimulation signal at said tip.

13. An endoscopic pedicle probe for use during spinal surgery to form a hole in a pedicle for reception of a pedicle screw, comprising:
an elongate shaft terminating in a distal tip that may be pushed through the pedicle to form the hole, and an enlarged proximal end for cooperation with the hand of the surgeon;
an endoscope and light extending through the shaft, said endoscope being connected with a monitor to enable a surgeon to visually observe the area being treated; and
a mechanomyographic monitoring system connected with the probe to perform dynamic pedicle integrity assessments during a procedure to detect a breach or potential breach of the pedicle and the proximity of the probe to a nerve and alert the surgeon so that a breach or contact with a nerve can be avoided.
